(19) United States
(12) Patent Application Publication
(10) Pub. No.: US 2010/0076561 A1
(43) Pub. Date: Mar. 25, 2010

(54) SET OF ELEMENTS TO BE ASSEMBLED IN ORDER TO FORM A SHOULDER PROSTHESIS

(75) Inventor: Mohan Emmanuel, Station Town Durham (GB)

Correspondence Address: YOUNG & THOMPSON, 209 Madison Street, Suite 500, Alexandria, VA, 22314, US

(73) Assignee: IMPLANTS INTERNATIONAL LIMITED, Cleveland Thorn-aby-on-tees (GB)

(21) Appl. No.: 12/442,229

(22) PCT Filed: Sep. 20, 2007
(86) PCT No.: PCT/IB2007/003776

§ 371 Date: Nov. 25, 2009

(30) Foreign Application Priority Data
Sep. 20, 2006 (FR) 06/08202

(51) Int. Cl. A61F 2/40

(52) U.S. Cl. 623/19.11

(57) ABSTRACT

A set includes at least one humeral element (2), at least one prosthetic head, and elements for mounting the prosthetic head in a plurality of positions in relation to the humeral element. The mounting elements include a plane and circular proximal face (12) of the humeral element, and a plate (13) projecting from the face and including indexing members, an inclined peripheral edge (15) and a groove (14). The set of elements includes at least one intermediary element including a base and a mounting end on one side of the base, the base having a cavity containing indexing members which complement those of the plate, and the cavity being dimensioned in such a way as to receive the plate in an adjusted manner. The base (21, 31) also includes a groove in the wall defining the periphery of the cavity, the groove receiving a split ring.

The present invention concerns a set of elements to be assembled in order to form a shoulder prosthesis.

It is well known to implant a shoulder prosthesis in order to recreate the shoulder joint, in particular following a fracture, comprising a humeral element and a prosthetic head. The humeral element has a portion in the form of a slender rod, designed to be introduced into the medullary cavity of the humerus, and proximal mounting means to mount the prosthetic head. The prosthesis can also comprise a glenoidal component designed to be anchored to the socket of the shoulder blade.

The prosthetic head must be positioned precisely in relation to the shoulder blade in order to obtain muscle tensions making it possible to cover the normal clearance of the joint. To this end, it is known to provide various types of mounting of the prosthetic head on the humeral element, allowing mounting of this head according to several different positions in relation to the humeral element.

The existing prostheses are not, however, fully satisfactory, either because they are limited as to the possibilities they allow for positioning of the prosthetic head, or because complete immobilization of the prosthetic head in relation to the humeral element is not perfectly ensured, or because the determination of the position of the prosthetic head in relation to the humeral element is not very easy to achieve.

The present invention aims to resolve this essential drawback, by providing a prosthesis which makes it possible to:
have very broad possibilities for positioning of the prosthetic head;
allow complete immobilization of the prosthetic head in relation to the humeral element;
allow easy determination of the position of the prosthetic head in relation to the humeral element.

The concerned set of elements comprises, in a known manner, at least one humeral element and at least one prosthetic head, the humeral element comprising means for fastening to the humerus, in particular in the form of a slender rod designed to be introduced into the medullary cavity of the humerus, and means for mounting the prosthetic head according to a plurality of positions in relation to the humeral element.

According to the invention,
said mounting means comprise a flat and circular proximal face of the humeral element and a projection projecting from this face, coaxially thereto, this projection being equipped with indexing means, with an inclined peripheral edge, at its free end, and with a groove arranged in its periphery;
the set of elements includes at least one intermediary element, comprising a base and, on one side of this base, a mounting end; the base has a circular shape defined, on the side opposite the end, by a flat face, and comprises a cavity arranged coaxially thereto, in which indexing means are formed which complement those comprised by said projection, this cavity being dimensioned in such a way as to receive said projection in an adjusted manner; the base also comprises a groove arranged in the wall defining the periphery of said cavity, receiving an elastically deformable split ring; this ring is normally shaped such that it extends partially in the groove of the base and partially outside this groove, and can be elastically deformed in the direction of an increase of its diameter so as to be completely retracted in this groove; the mounting end is secured to said base and is arranged non-coaxially thereto, this end being configured to receive and retain the prosthetic head;
the prosthetic head comprises, at its axis, a cavity for receiving the mounting end; the set is such that said intermediary element can be mounted on said humeral element by engagement of said projection in said cavity, according to a plurality of angular positions around the axis of the projection and of the cavity thanks to respective indexing means comprised by this projection and this cavity; during this engagement, said inclined peripheral edge performs said elastic deformation of the ring, which allows the passage of this projection beyond the ring; the arrival of the respective flat faces of the humeral element and of the base in immediate proximity to each other coincides with the arrival of said groove across from the ring; in this position, the latter resumes its normal shape through elastic return and as a result partially engages inside this groove, ensuring mounting of the intermediary element on the humeral element.

Thus, the prosthesis obtained by the set of elements according to the invention comprises an intermediary element capable of being mounted according to a plurality of angular positions on the proximal end of the humeral element, with the arrival of the peripheral edge of the base of the intermediary element coinciding with the peripheral edge of the proximal flat face of the humeral element and the arrival of the flat face comprised by this intermediate element in contact with the corresponding flat face comprised by the humeral element, and with locking of the projection in the cavity via the split ring. The set of elements according to the invention, thus configured, makes it possible to form the proximal part of the humeral component of the prosthesis having a constant shape regardless of the angular position of the intermediary element on the humeral element, with complete immobilization of the prosthetic head in relation to the humeral element.

The set of elements can comprise at least one intermediary element of a first type, whereof the axis of the mounting end is parallel to the axis of the cavity.

This intermediate element allows adjustment of the position of the prosthetic head according to orbital positions distributed over directions perpendicular to the axis of the projection of the humeral element.

The set of elements can comprise several intermediary elements of said first type, the mounting ends of which are located, from one intermediary element to another, at different distances from the axis of the cavities of these elements, thereby making it possible to increase the possibilities for adjusting the position of the prosthetic head.

The set of elements can also comprise at least one intermediary element of a second type, whereof the axis of the mounting end forms an angle with the axis of the cavity of this intermediate element.

This second intermediary element allows adjustment of the incline position of the prosthetic head in relation to the axis of the projection of the humeral element.

In the same manner as before, the set of elements can comprise several intermediary elements of said second type, the mounting ends of which have, from one intermediary element to another, different inclines in relation to the axis of the cavities, also making it possible to increase the possibilities for adjusting the position of the prosthetic head.

The set of elements according to the invention can also comprise at least one intermediary element of a third type, combining the characteristics of an intermediary element of said first and said second types, i.e. whereof the axis of the mounting end is both offset in relation to the axis of the cavity of this intermediary element and inclined in relation to the axis of this same cavity.

According to one preferred embodiment of the invention, said indexing means are formed by channels arranged in said projection and in said cavity. In particular, the projection and the cavity can comprise twelve channels, allowing twelve possible angular positions of an intermediary element in relation to the humeral element.

Advantageously, the humeral element comprises a reference mark, for example at the median plane of this element, on the external side, and at least one intermediary element comprises, at the periphery of said base, at least one mark corresponding to a determined angular position of this intermediary element in relation to the humeral element.

The determination of a position of the prosthetic head is thus particularly easy to achieve.

The invention will be well understood, and other characteristics and advantages thereof will appear, in reference to the appended diagrammatic drawing, this drawing showing, as a non-limiting example, one preferred embodiment of several elements of the set of elements it concerns.

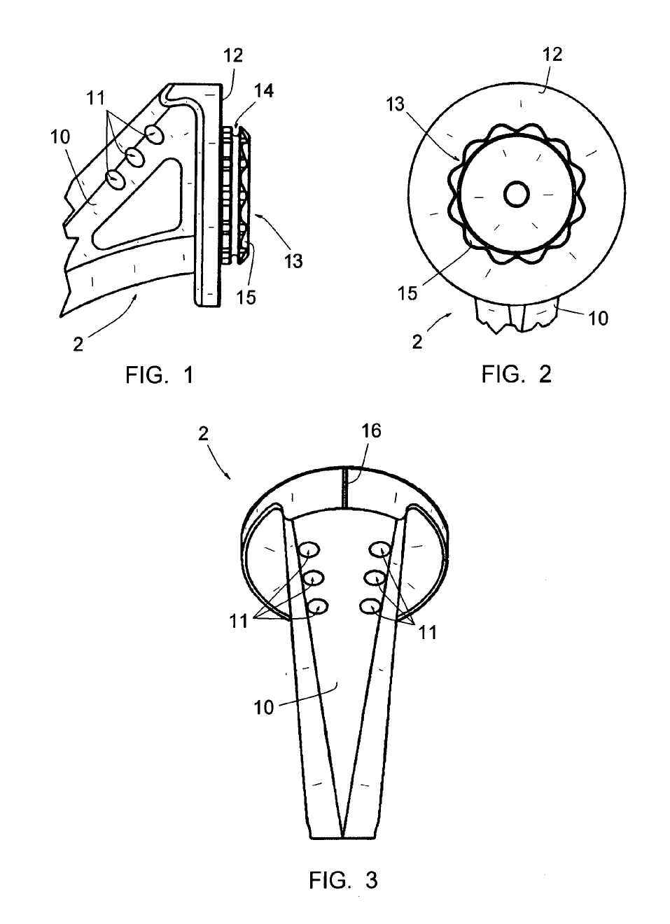

FIG. 1 is a partial side view of a humeral element comprised by this set;

FIG. 2 is an end view, from the proximal side, along the axis of a proximal end face comprised by this element;

FIG. 3 is a lateral view of its external side;

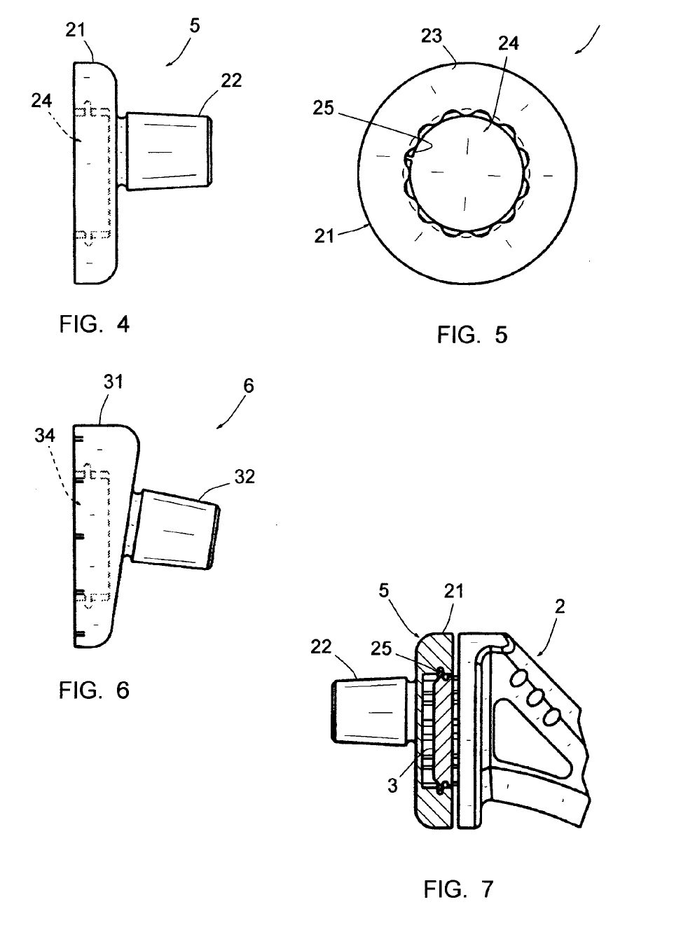

FIG. 4 is a side view of an intermediary element comprised by the set of elements;

FIG. 5 is a view of this same element from its distal side;

FIG. 6 is a side view of another intermediary element comprised by the set of elements;

FIG. 7 is a partial side view, partially in cross-section, of the intermediary element of FIG. 4 during mounting on the humeral element of FIG. 1;

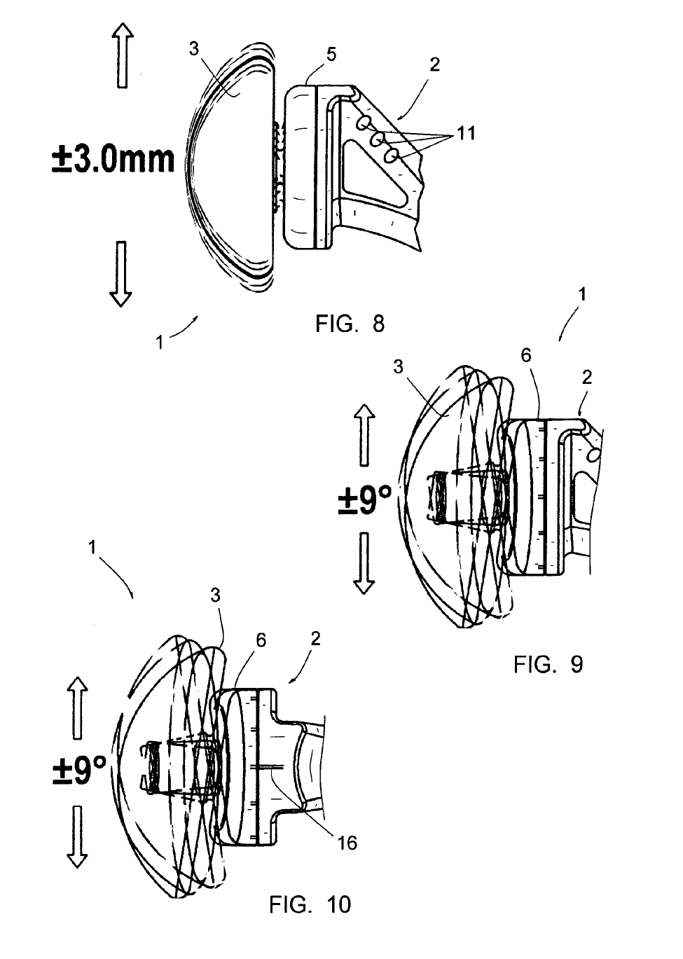

FIG. 8 is a partial view of the two elements of FIG. 7 after mounting and of a prosthetic head connected thereto, several possible positions of this head being shown in broken lines;

FIG. 9 is a partial side view of the intermediate element of FIG. 6 mounted on the humeral element of FIG. 1 and of the prosthetic head connected to them, several possible positions of this head being shown in broken lines;

FIG. 10 is a view similar to that of FIG. 9, from the top; and

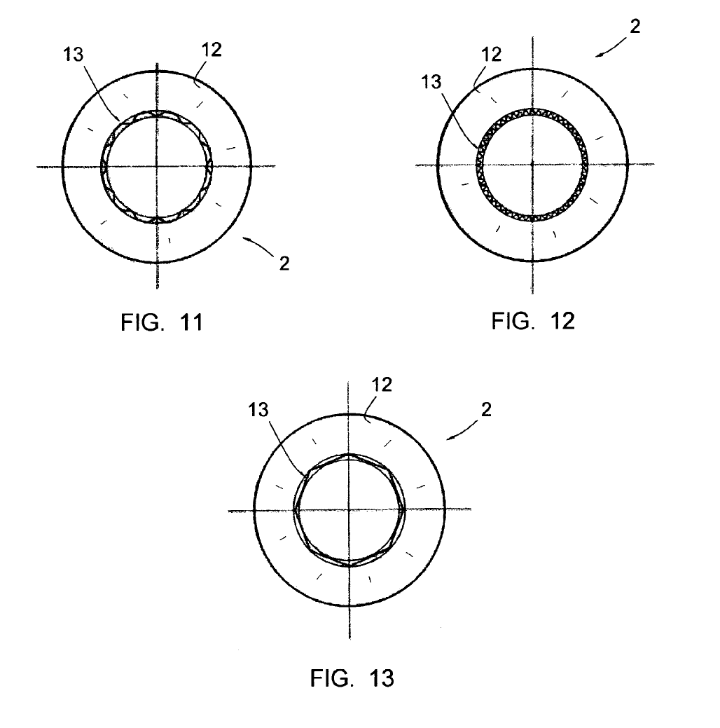

FIGS. 11 to 13 are views of three variations of embodiment of the abovementioned elements.

FIGS. 8 to 10 show a shoulder prosthesis 1 comprising a humeral element 2 and a prosthetic head 3. The humeral element 2 is shown more particularly by FIGS. 1 to 3 and can receive one or the other of the intermediary elements 5 or 6 shown more particularly by FIGS. 4 and 6.

The prosthesis 1 can also comprise a glenoidal element designed to be anchored to the socket of the shoulder blade.

In reference to FIGS. 1 to 3, the humeral element 2 has a portion 10 in the form of a slender rod, designed to be introduced into the medullary cavity of the humerus, which can comprise, as in the illustrated example, holes 11 for the passage of wires for maintaining tuberosities.

The element 2 has a circular proximal flange, forming a flat and circular proximal face 12, from which a projection 13 projects, coaxially thereto. The projection 13 has, at its periphery, a series of twelve channels defining radial protrusions between them, the set forming indexing means. The projection 13 also has, substantially at mid-height, a peripheral groove 14, and, at its free end, an inclined peripheral edge 15.

FIG. 3 also shows that the proximal flange comprises a reference mark 16, printed on its edge, at the median plane of the element 2, on the external side thereof.

FIGS. 4 and 5 show a first intermediary element 5, comprising a base 21 and, on one side of this base 21, an end 22 for mounting the head 3.

The base 21 has a circular shape defined, on the side opposite the end 22, by a flat face 23, and comprises a cavity 24 arranged coaxial thereto. In the wall defining the periphery of this cavity are arranged twelve channels defining protrusions between them, the set forming indexing means which complement those comprised by the projection 13. The cavity 24 is dimensioned so as to receive the projection in an adjusted manner. The base 21 also comprises a groove arranged in the wall defining the periphery of the cavity 24, receiving an elastically deformable split ring 25. This ring 25 normally has a shape such that it extends partially in the groove of the base 21 and partially outside this groove, and can be elastically deformed in the direction of an increase of its diameter so as to be completely retracted in this groove.

The end 22 is secured to the base 21. It has a Morse taper shape, and its axis is radially offset in relation to that of the cavity 24 while being parallel to the axis of said cavity.

FIG. 6 shows the second intermediary element 6, comprising a base 31 and an end 32 for mounting the head 3. The base 31 is quasi-identical to the base 21; in addition to the latter, it comprises reference marks on its edge, designed to cooperate with the mark 16 so as to enable identification of the different possible angular positions of the element 6 in relation to the element 2.

The end 32 has a same shape as the end 22 and allows mounting of the head 3 in the same way. Unlike the end 22, the axis of the end 32 forms an angle with the axis of the cavity 34 of the element 6. This angle is 9° in the illustrated example.

The prosthetic head 3 comprises, at its axis, a cavity for receiving one or the other of the mounting ends 22, 32.

As will be understood in reference to FIGS. 7 to 10, the set of elements according to the invention is such that one or the other of said intermediary elements 5, 6 can be mounted on the humeral element 2 by engagement of said projection 13 in the corresponding cavity 24, 34, according to a plurality of angular positions around the axis of the projection 13 and of the cavity 24, 34 thanks to respective indexing means comprised by said projection 13 and cavity 24, 34; during this engagement, the inclined peripheral edge 15 performs said elastic deformation of the ring 25, which allows the passage of this projection 13 beyond the ring 25; the arrival of the respective flat faces 12, 23 of the humeral element 2 and of the base 21, 31 in immediate proximity to each other coincides with the arrival of said groove 14 opposite the ring 25; in this position, the latter resumes its normal shape by elastic return and as a result engages partially inside this groove 14, ensuring mounting of the intermediary element 5, 6 on the humeral element 2.

The prosthesis obtained by the set of elements according to the invention therefore comprises an intermediary element 5, 6 able to be mounted according to a plurality of angular positions on the proximal end of the humeral element, with the peripheral edge of the base 21, 31 of this intermediary element 5, 6 coinciding with the peripheral edge of the proximal flat face 12 of the humeral element, and arrival of the flat face 23 comprised by this intermediary element 5, 6 in contact with the corresponding flat face comprised by the humeral element 2, and with locking of the projection 13 in the cavity 24, 34 using the split ring 25. The set of elements according to the invention, thus configured, makes it possible to form a proximal part of the humeral component of the prosthesis having a constant shape regardless of the angular position of the intermediary element 5, 6 on the humeral element 2, with complete immobilization of the prosthetic head 3 in relation to the humeral element 2.

As shown by FIG. 8, the intermediary element 5 allows, depending on its angular position in relation to the element 2, adjustment of the position of the prosthetic head 3 according to twelve orbital positions distributed over directions perpendicular to the axis of the projection 13.

FIGS. 9 and 10 show that the intermediary element 6 allows, depending on its angular position in relation to the element 2, adjustment of the incline position of the prosthetic head in relation to the axis of the projection 13.

FIGS. 11 to 13 show different variations of the respective indexing means comprised by the projection 13 and the cavity 24, 34: “gear tooth” channels (FIG. 11), triangular teeth (FIG. 12) or facets (FIG. 13).

It goes without saying that the invention is not limited to the embodiment described above as an example, but that it extends to all embodiments covered by the appended claims.

1. A set of elements to be assembled in order to form a shoulder prosthesis (1), comprising at least one humeral element (2) and at least one prosthetic head (3), the humeral element (2) comprising means for fastening to the humerus, in particular in the form of a slender rod (10) designed to be introduced into the medullary cavity of the humerus, and mounting means (3, 5, 6, 12, 23, 24, 25) for mounting the prosthetic head (3) according to a plurality of positions in relation to the humeral element, characterized in that
said mounting means comprise a flat and circular proximal face (12) of the humeral element (2) and a projection (13) projecting from this face (12), coaxially thereto, this projection (13) being equipped with indexing means, with an inclined peripheral edge (15), at its free end, and with a groove (14) arranged in its periphery;
the set of elements includes at least one intermediary element (5, 6), comprising a base (21, 31) and, on one side of this base (21, 31), a mounting end (22, 32); the base (21, 31) has a circular shape defined, on the side opposite the end (22, 32), by a flat face (23), and comprises a cavity (24, 34) arranged coaxially thereto, in which indexing means are formed which complement those comprised by said projection (13), this cavity (24, 34) being dimensioned in such a way as to receive said projection (13) in an adjusted manner; the base (21, 31) also comprises a groove arranged in the wall defining the periphery of said cavity (24, 34), receiving an elastically deformable split ring (25); this ring (25) is normally shaped such that it extends partially in the groove of the base (21, 31) and partially outside this groove, and can be elastically deformed in the direction of an increase of its diameter so as to be completely retracted in this groove; the mounting end (22, 32) is secured to said base (21, 31) and is arranged non-coaxially thereto, this end (22, 32) being configured to receive and retain the prosthetic head (3);
the prosthetic head (3) comprises, at its axis, a cavity for receiving the mounting end (22, 32);
the set is such that said intermediary element (5, 6) can be mounted on said humeral element (2) by engagement of said projection (13) in said cavity (24, 34), according to a plurality of angular positions around the axis of the projection (13) and of the cavity (24, 34) thanks to respective indexing means comprised by this projection (13) and this cavity (24, 34); during this engagement, said inclined peripheral edge (15) performs said elastic deformation of the ring (25), which allows the passage of this projection (13) beyond the ring (25); the arrival of the respective flat faces (12, 23) of the humeral element (2) and of the base (21, 31) in immediate proximity to each other coincides with the arrival of said groove (14) across from the ring (25); in this position, the latter resumes its normal shape through elastic return and as a result partially engages inside this groove (14), ensuring mounting of the intermediary element (5, 6) on the humeral element (2).

2. The set of elements according to claim 1, characterized in that it comprises at least one intermediary element (5) of a first type, whereof the axis of the mounting end (22) is parallel to the axis of the cavity (24).

3. The set of elements according to claim 2, characterized in that it comprises several intermediary elements (5) of said first type, whereof the mounting ends (22) are located, from one intermediary element (5) to another, at different distances from the axis of the cavities (24), thereby making it possible to increase the possibilities for adjusting the position of the prosthetic head (3).

4. The set of elements according to claim 1, characterized in that it comprises at least one intermediary element (6) of a second type, whereof the axis of the mounting end (32) forms an angle with the axis of the cavity (34) of this intermediary element.

5. The set of elements according to claim 4, characterized in that it comprises several intermediary elements (6) of said second type, whereof the mounting ends (32) have, from one intermediary element (6) to another, different inclines in relation to the axis of the cavities (34), also making it possible to increase the possibilities for adjusting the position of the prosthetic head (3).

6. The set of elements according to claim 2, characterized in that it comprises at least one intermediary element of a third type, combining the characteristics of an intermediary element of said first and said second types, i.e. whereof the axis of the mounting end is both offset in relation to the axis of the cavity of this intermediary element and is inclined in relation to the axis of this same cavity.

7. The set of elements according to claim 1, characterized in that said indexing means are formed by channels arranged in said projection (13) and in said cavity (24, 34).

8. The set of elements according to claim 1, characterized in that the humeral element (2) comprises a reference mark (16), for example at the median plane of this element, on the external side, and in that at least one intermediary element (6) comprises, at the periphery of said base (31), at least one mark corresponding to a determined angular position of this intermediary element (6) in relation to the humeral element (2).

9. The set of elements according to claim 2, characterized in that it comprises at least one intermediary element (6) of a second type, whereof the axis of the mounting end (32) forms an angle with the axis of the cavity (34) of this intermediary element.

10. The set of elements according to claim 3, characterized in that it comprises at least one intermediary element (6) of a second type, whereof the axis of the mounting end (32) forms an angle with the axis of the cavity (34) of this intermediary element.

11. The set of elements according to claim 3, characterized in that it comprises at least one intermediary element of a third type, combining the characteristics of an intermediary element of said first and said second types, i.e. whereof the axis of the mounting end is both offset in relation to the axis of the cavity of this intermediary element and is inclined in relation to the axis of this same cavity.

12. The set of elements according to claim 4, characterized in that it comprises at least one intermediary element of a third type, combining the characteristics of an intermediary element of said first and said second types, i.e. whereof the axis of the mounting end is both offset in relation to the axis of the cavity of this intermediary element and is inclined in relation to the axis of this same cavity.

13. The set of elements according to claim 5, characterized in that it comprises at least one intermediary element of a third type, combining the characteristics of an intermediary element of said first and said second types, i.e. whereof the axis of the mounting end is both offset in relation to the axis of the cavity of this intermediary element and is inclined in relation to the axis of this same cavity.
